(19) United States
(12) Patent Application Publication
(10) Pub. No.: US 2010/0068740 A1
(43) Pub. Date: Mar. 18, 2010

(54) MICROFLUIDIC DEVICE WITH A CYLINDRICAL MICROCHANNEL AND A METHOD FOR FABRICATING SAME

(75) Inventors: David L. Kaplan, Concord, MA (US); Fiorenzo Omenetto, Wakefield, MA (US); Brian Lawrence, New York, NY (US); Mark Cronin-Golomb, Reading, MA (US); Irene Georgakoudi, Acton, MA (US); Hannah Perry, Kingston, RI (US)

Correspondence Address: DAVID S. RESNICK, NIXON PEABODY LLP, 100 SUMMER STREET, BOSTON, MA, 02110-2131, US

(73) Assignee: TRUSTEES OF TUFTS COLLEGE, Medford, MA (US)

(21) Appl. No.: 12/513,416

(22) PCT Filed: Nov. 5, 2007
(86) PCT No.: PCT/US07/83646

§ 371 Date: May 4, 2009

Related U.S. Application Data
(60) Provisional application No. 60/856,297, filed on Nov. 3, 2006.
(60) Provisional application No. 60/906,509, filed on Mar. 13, 2007.

(51) Int. Cl. C12M 1/34; B29C 39/02; C12Q 1/02; C12Q 1/28; C12Q 1/54; C12Q 1/34

(52) U.S. Cl. 435/14; 264/236; 435/288.7; 435/29; 435/28; 435/18

(57) ABSTRACT

A method of manufacturing a microfluidic device having at least one cylindrical microchannel includes providing a substrate, casting an uncured polymer matrix solution onto the substrate, embedding an elongated rod in the uncured polymer matrix solution, curing the polymer matrix solution to form a solidified body, and extracting the elongated rod to form the cylindrical microchannel in the solidified body. In another embodiment, the method includes forming an optical feature on a surface of the microfluidic device. A microfluidic device is also provided, the device including a polymer body, and at least one cylindrical microchannel in the polymer body, the cylindrical microchannel having a diameter between approximately 40 ?m and 250 ?m, inclusive. An additional microfluidic device is provided that functions as an optofluidic spectrometer. The optofluidic spectrometer includes a polymer body, a diffraction grating integrated within the polymer body, and a cylindrical microchannel behind the diffraction grating on the polymer body.

### CROSS-REFERENCE TO RELATED APPLICATIONS

This application claims the benefit of priority of U.S. Provisional Application Ser. No. 60/856,297 filed on Nov. 3, 2006, entitled “Biopolymer Devices and Methods for Manufacturing the Same” and to U.S. provisional Application Ser. No. 60/906,509 filed on Mar. 13, 2007.

### GOVERNMENT SUPPORT

The invention was made with government support under FA95500410363 awarded by the Air Force Office of Scientific Research. The government has certain rights in this invention.

### BACKGROUND OF THE INVENTION

1. Field of the Invention

The present invention is directed to a microfluidic device having a cylindrical microchannel and a method of fabricating such a microfluidic device.

2. Description of Related Art

Microfluidic devices having three dimensional (3-D) microchannels for conveying fluid and methods for manufacturing such devices are known in the art. The functionality of polymer-based microfluidic devices has recently made these microfluidic devices an important resource for the scientific community. Such devices hold great promise in the field of biomedical engineering by combining small features, sized from 10 μm to 200 μm, with the ability to accommodate biological samples.

Conventional microfluidic devices with microchannel geometries are usually prepared by using soft-lithography techniques to form semi-circular surface channels on a surface of a polymer film. Two polymer films with such surface channels are then stacked together with their semi-circular surface channels facing each other. When assembled in this fashion, the two polymer films together define a cylindrical microchannel. However, to provide a cylindrical microchannel in this conventional manner requires precise stacking, aligning, and fixing of the semi-circular surface channels of the films Thus, the ability to provide a cylindrical microchannel is directly impacted by material and manufacturing tolerances and precise stacking, aligning, and fixing of the semi-circular surface channels of the films

Correspondingly, the difficulties associated with precise stacking, aligning, and fixing the films renders this conventional method of producing microfluidic devices inefficient for providing microchannels with a cylindrical cross-section. The necessity for precise alignment of the films is compounded as the diameter of the microchannel becomes smaller. For example, microchannels having a diameter of less than 60 μm and smaller require precision in stacking, aligning, and fixing the films that is extremely difficult. Even when such precise positioning of the films can be attained, the assembly of film halves in this manner results in a seam in the microchannel. This seam in the microchannel can affect fluidic flow through the channel. Additionally, air bubbles can form between the surfaces of the mating films during conventional manufacturing presenting further difficulties and limitations in conventional fabrication of the channels. Yet another complication and difficulty in fabricating microfluidic devices using the described conventional method is that the two films must be extremely flat to properly mate together to form the microchannel. Otherwise, gaps along the seam in the microchannel can form, which further impact fluid flow through the channel.

For example, a primary and important disadvantage of the above-described conventional method of manufacturing a microfluidic device with microchannels is shown in FIG. 1. As can be seen, the microfluidic device 1 includes a first film 3 having a semi-circular surface channel 4 formed on a surface and a second film 7 having a semi-circular surface channel 8 formed on its surface. As explained above, the first film 3 and the second film 7 are stacked together with their respective semi-circular surface channels 4 and 8 facing each other in the manner shown in FIG. 1, so as to form the microchannel 9 embedded in the microfluidic device 1.

In theory, the microchannel 9 would have a circular cross section, and the microchannel 9 would be cylindrical in shape as it extends in and out of the page in the illustration of FIG. 1. However, as can be seen, the difficulties associated with precise alignment of the first film 3 and the second film 7 causes misalignment of the semi-circular surface channels 4 and 8, thereby resulting in a microchannel that does not have a circular cross section. In this regard, as can also be appreciated, the tolerances and positioning inaccuracies can be greater than the size of the microstructure itself when the microchannel is very small, for example, 40 μm. Thus, as shown in FIG. 1, the microchannel 9 that is defined by the two films 3, 7 of the microfluidic device 1 is not a cylindrical microchannel.

Non-cylindrical microchannels may be sufficient for certain applications, but such non-cylindrical microchannels do not resemble naturally-occurring fluidic microchannels typically found in microvasculature of animals and humans. In this regard, the non-cylindrical geometry significantly impacts the flow characteristics of fluids, such as blood, conveyed through the microchannel. Thus, the above described method of providing a microfluidic device is not suitable for modeling microvasculature in animals and humans, and is not suitable for biomedical applications where a cylindrical microchannel is desirable.

Laser ablation techniques have also been shown to be effective for forming embedded microchannels with diameters of a few microns or smaller. However, forming larger diameter microchannels in the range of approximately 40 μm to 250 μm would require larger beams and larger fluence. In addition, using laser ablation techniques for such larger diameter microchannels poses problems in disposing the debris generated by the ablation process.

Correspondingly, there exists an unfulfilled need for a microfluidic device that has one or more cylindrical microchannels. There also exists an unfulfilled need for a method of fabricating a microfluidic device having one or more microchannels therein that avoids the limitations of the conventional techniques described above. In addition, there still exists an unfulfilled need for a method of forming one or more cylindrical microchannels that can be used to model microvasculature of animals and humans.

### SUMMARY OF THE INVENTION

In view of the foregoing, an aspect of the present invention is in providing a microfluidic device with at least one microchannel.

Another aspect of the present invention is in providing a method for forming one or more cylindrical microchannels.

An advantage of the present invention is in providing a method for fabricating a microfluidic device with one or more cylindrical microchannels that can be used to model microvasculature of animals and humans.

An advantage of the present invention is in combining photonic and microfluidic devices to create geometries with additional functionality, compactness, and enhanced integration. A microfluidic device in accordance with the present invention that incorporates photonically significant geometries such as fiber waveguides, photonic crystals, and the like, allows fluids to infiltrate the devices and to modify the local optical environment of the device. Further, the microfluidic device may be modified in this fashion to provide tunability that did not exist in the original photonic structure. Additional tunability features may be incorporated by varying the chemical and optical properties of the fluid itself. Conversely, the nature and composition of the fluid may be determined by observing the response of a photonic structure with known behavior. These optofluidic structures may perform optical sensing.

In the above regard, a method of manufacturing a microfluidic device having at least one cylindrical microchannel is provided in accordance with one aspect of the present invention. In one embodiment, the method includes providing a substrate, casting an uncured polymer matrix solution onto the substrate, embedding an elongated rod in the uncured polymer matrix solution, curing the polymer matrix solution to form a solidified body of the microfluidic device, and extracting the elongated rod to form the cylindrical microchannel in the solidified body. In this regard, the method may also include suspending the elongated rod over the substrate.

In one embodiment, the elongated rod is a silica rod having a diameter between approximately 40 μm and 250 μm, for example between approximately 57 μm and 125 μm. In one embodiment, the biopolymer matrix solution is a silk fibroin matrix solution having approximately 1.0 wt % to 30 wt % silk, inclusive. For example, the silk fibroin matrix solution may have approximately 8.0 wt % silk. In another embodiment, the polymer matrix solution is polydimethylsiloxane (PDMS). In another embodiment, the polymer matrix solution is a biopolymer such as chitosan, collagen, gelatin, agarose, chitin, polyhydroxyalkanoates, pullan, starch (amylose amylopectin), cellulose, hyaluronic acid, and related biopolymers, or variations or combinations thereof. In still another embodiment, the method of the present invention may further include applying heat to the uncured polymer matrix solution to cure the solution. In addition, the method may further include coating the silica rod with a surfactant solution.

In yet another embodiment, the method of the present invention may include forming an optical element on a surface of the microfluidic device or upon a substrate. In this regard, the substrate may be a template for an optical element such as a lens, a microlens array, an optical grating, a pattern generator, a beam reshaper, a mirror blank, or a glass slide. In another embodiment, the method may further include adding a doping agent to the uncured polymer matrix solution, where the doping agent may be an organic material such as red blood cells, horseradish peroxidase, phenolsulfonphthalein, or a combination thereof. The organic material can also be a nucleic acid, a dye, a cell, an antibody, enzymes, for example, peroxidase, lipase, amylose, organophosphate dehydrogenase, ligases, restriction endonucleases, ribonucleases, DNA polymerases, glucose oxidase, laccase, cells, viruses, proteins, peptides, small molecules, drugs, dyes, amino acids, vitamins, antixoxidants, DNA, RNA, RNAi, lipids, nucleotides, aptamers, carbohydrates, chromophores, light emitting organic compounds such as luciferin, carotenes and light emitting inorganic compounds, chemical dyes, antibiotics, antifungals, antivirals, light harvesting compounds such as chlorophyll, bacteriorhodopsin, protorhodopsin, and porphyrins and related electronically active compounds, or a combination thereof.

In accordance with another aspect of the present invention, a microfluidic device is provided, the device comprising a polymer body and at least one cylindrical microchannel in the polymer body where the cylindrical microchannel has a diameter between approximately 40 μm and 250 μm, inclusive. For example, the cylindrical microchannel may have a diameter between approximately 57 μm and 125 μm, inclusive. The polymer body may be made of polydimethylsiloxane (PDMS) in one embodiment, but in other embodiments the polymer body may be made of a biopolymer such as silk, chitosan, collagen, gelatin, agarose, chitin, polyhydroxyalkanoates, pullan, starch (amylose amylopectin), cellulose, hyaluronic acid, and related biopolymers, or a combination or variation thereof. In addition, the polymer body may be implemented to include a doping agent and an optical element on a surface of the polymer body. The doping agents may include organic materials such as red blood cells, horseradish peroxidase, and phenolsulfonphthalein, for example. The optical elements may include a lens, a microlens array, an optical grating, a pattern generator, a beam reshaper, a mirror blank, and a glass slide. The organic material can also be a nucleic acid, a dye, a cell, an antibody, enzymes, for example, peroxidase, lipase, amylose, organophosphate dehydrogenase, ligases, restriction endonucleases, ribonucleases, DNA polymerases, glucose oxidase, laccase, cells, viruses, proteins, peptides, small molecules, drugs, dyes, amino acids, vitamins, antixoxidants, DNA, RNA, RNAi, lipids, nucleotides, aptamers, carbohydrates, chromophores, light emitting organic compounds such as luciferin, carotenes and light emitting inorganic compounds, chemical dyes, antibiotics, antifungals, antivirals, light harvesting compounds such as chlorophyll, bacteriorhodopsin, protorhodopsin, and porphyrins and related electronically active compounds, or a combination thereof.

In another embodiment of the present invention, coupled microfluidic structures are used to perform biochemical reactions and analysis on a planar substrate. Using the microfluidic device and method of the present invention, these reactions typically requiring only pico-liters of reagents. The devices may be mass produced and employ a standardized reaction vessel that only uses small quantities of samples and analytes, when applied to pathology for example. The devices and methods allow many tests to be run in parallel from a single sample, thereby reducing costs.

Additionally, optical sensing functionalities are integrated to provide greater diagnostic versatility than previously possible. One such optical functionality is that of spectroscopy. By incorporating a spectroscopic device in accordance with the present invention, the absorption spectra of an analyte may be determined to provide a measure of concentration of species, contaminant levels, and other measures.

These and other features and advantages of the present invention will become more apparent from the following detailed description of the preferred embodiments of the present invention when viewed in conjunction with the accompanying drawings.

### BRIEF DESCRIPTION OF THE DRAWINGS

FIG. 1 schematically illustrates a cross-sectional view of a microfluidic device with a microchannel that is fabricated using a conventional method.

FIGS. 2A through 2C schematically illustrate a method of forming a microfluidic device in accordance with one embodiment of the present invention.

FIG. 3 is a photograph of a microfluidic device manufactured in accordance with one embodiment of the present invention.

FIG. 4A is a scanning electron microscope image of an orthogonal section of a 125 μm diameter microchannel manufactured in accordance with one embodiment of the present invention.

FIG. 4B is a scanning electron microscope image of a longitudinal section of the microchannel shown in FIG. 4A.

FIG. 5A is a scanning electron microscope image of an orthogonal section of a 57 μm diameter microchannel in manufactured in accordance with one embodiment of the present invention.

FIG. 5B is a scanning electron microscope image of a longitudinal section of the microchannel shown in FIG. 5B.

FIG. 6A is an enlarged still frame image showing heparin in the blood flowing through the microchannel of the microfluidic device shown in FIG. 3.

FIG. 6B is an enlarged still frame image showing erythrocytes in the blood flowing through the microchannel of the microfluidic device shown in FIG. 3.

FIG. 7A is a graph showing the detection of red blood cells flowing in the microchannel of the microfluidic device shown in FIG. 3.

FIG. 7B is a graph showing a base output while a medium flows through the microchannel of the microfluidic device shown in FIG. 3.

FIG. 8 schematically illustrates a microfluidic device for use as a scanning grating spectrometer in accordance with the present invention.

FIG. 9 shows a schematic illustration of the experimental setup of the scanning grating spectrometer of FIG. 8.

FIG. 10A is a graph of the absorption spectrum over various wavelengths as measured by the scanning grating spectrometer device of FIGS. 8 and 9.

FIG. 10B is a graph of the temporal response of the scanning grating spectrometer device of FIGS. 8 and 9 at a wavelength of 660 nm.

### DETAILED DESCRIPTION OF THE PREFERRED EMBODIMENT

In accordance with one embodiment of the present invention, cylindrical channels may be formed in a polymer using rods of controllable diameter. The rods may be fixed upon mounts or specific molds and may be held in place using adhesive films. An uncured polymer solution or biopolymer matrix solution may be deposited onto the molds to immerse the rods. The uncured polymer is then cured. The curing is performed at temperatures to avoid distortion of the rods. The matrix polymerizes, and the solidified matrix is subsequently removed from the mold. The silica rods are extracted, and the result is a highly regular, cylindrical microchannel within the polymer.

FIGS. 2A to 2C schematically illustrate a method of fabricating a microfluidic device in accordance with one embodiment of the present invention where silica rods of a selected diameter are used to form the cylindrical microchannel in the microfluidic device. More specifically, in accordance with a method of the present invention, an uncured polymer matrix solution 10 made of a polymer or a biopolymer is cast onto an appropriate substrate 12. An elongated, cylindrical rod 14 or wire is embedded in the uncured polymer matrix solution 10 so that the cylindrical rod 14 is surrounded by the uncured polymer matrix solution 10 and positioned over substrate 12.

The elongated rod 14 or wire in the illustrated implementation of FIGS. 2B and 2C may be a silica rod 14, such as a silicon fiber used in the optical fiber industry. Likewise, other materials may also be used for the elongated rod 14. The elongated rod 14 may be secured on mounts and held in place using adhesive films, fixed metallic spacers, or other appropriate mechanical retaining devices, so that the elongated rod 14 maintains its shape as it is embedded in the uncured polymer matrix solution 10 over the substrate 12. Alternatively, the silica rod 14 may be appropriately secured to mounts so that it is suspended over substrate 12, and the uncured polymer matrix solution 10 is cast over the substrate 12 and the silica rod 14 until the silica rod 14 is completely immersed in the uncured polymer matrix solution 10. The substrate 12 may be any appropriate mold that can be used as a substrate, such as an optical device, including the optical grating schematically shown in FIG. 2A.

The uncured polymer matrix solution 10 utilized for the formation of the microfluidic device may be polydimethylsiloxane (PDMS), the silica rod 14 possessing adequate strength to withstand submersion within the uncured PDMS solution. Of course, other polymers including biopolymers such as silk, chitosan, collagen, gelatin, agarose, chitin, polyhydroxyalkanoates, pullan, starch (amylose amylopectin), cellulose, hyaluronic acid, and related biopolymers, or a combination thereof, may be used in other implementations. The use of PDMS for the fabrication of the microfluidic device is especially advantageous in that flow through the microchannel can be easily inspected.

The polymer matrix solution 10 with the embedded silica rod 14 as shown in FIG. 2B is then polymerized to form a solidified body 16 of the microfluidic device. The polymer matrix solution 10 may be oven cured, depending on the polymer matrix solution 10 used. However, curing temperatures should be less than the distortion temperatures of the silica rod 14 to avoid geometric and structural distortion of the silica rod 14, which can make extraction difficult. Specifically, for the PDMS utilized in the present example discussed herein, the polymer matrix solution 10 is oven cured at approximately 115° C. This curing temperature of the polymer matrix solution 10 is substantially less than the softening temperature of the silica glass of silica rod 14, which is greater than 2000° C., thereby ensuring mechanical stability of the silica rod 14 during fabrication of the microfluidic device.

Upon polymerization of the matrix solution 10, the silica rod 14 is then extracted from the solidified body 16 of the microfluidic device, and the solidified body 16 is removed from the mold, for example the substrate 12 as shown in FIG. 2C. To extract the silica rod 14 from the solidified body 16, one end of the silica rod 14 is pulled out of the solidified body 16 of the microfluidic device as shown in FIG. 2C. In the example where PDMS is utilized for the polymer matrix solution 10, the silica rod 14 is strong enough to allow the silica rod 14 to be simply pulled out by mechanical force. However, if other matrix solutions are used, such as biopolymers, for example, the extraction of the silica rod 14 may be facilitated by coating the silica rod 14 with a surfactant solution. The surfactant solution reduces adhesion between the silica rod 14 and the cured matrix solutions, if necessary. Upon polymerization of the matrix solution 10 and extraction of the silica rod 14, the resulting microfluidic device 20 includes a highly regular, cylindrical microchannel 22 extending through the microfluidic device 20.

Additionally, by forming the microfluidic device 20 from PDMS in one embodiment, the cylindrical microchannel 22 of the microfluidic device 20 serves as an embedded optical element to allow various spectral flow studies. In particular, as noted above, the microfluidic device 20 is transparent so that optical studies can be performed on a fluid as the fluid is conveyed through the cylindrical microchannel 22. In addition, the substrate 12, which serves as a mold in the present example, is an optical device such as an optical grating, which is designed to incorporate specific optical features on the surface of the body of the microfluidic device 20 to provide additional functionality to the microfluidic device 20. In other embodiments, the base surface onto which the polymer matrix solution is cast may be a lens, a microlens array, a pattern generator, a beam reshaper, and the like. Additionally, the base surface may also be glass substrates, such as mirror blanks, or glass slides that are substantially smooth to allow formation of a high-quality optical surface on the microfluidic device 20. While formation of a single cylindrical microchannel is shown and described as an example above, the present invention may be also used to provide a plurality of cylindrical microchannels through microfluidic devices as well.

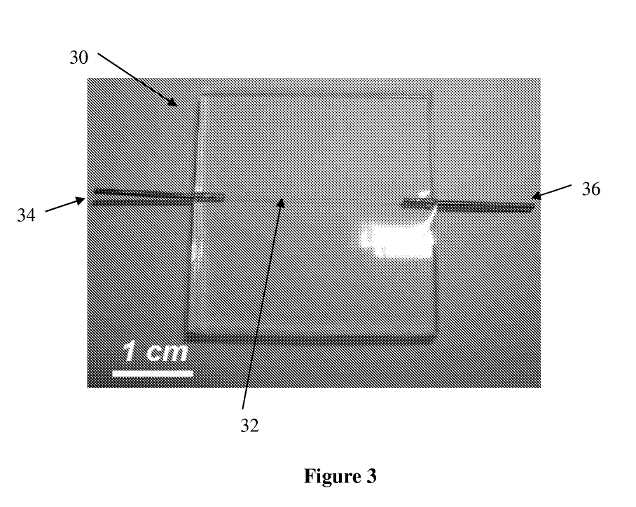

FIG. 3 is a photograph of a microfluidic device 30 fabricated in accordance with a method of the present invention described above. The microfluidic device 30 was manufactured using PDMS, for example PDMS that is available from GE Silicones under the name RTV615. The microfluidic device 30 includes a cylindrical microchannel 32, which is 57 μm in diameter and approximately 300 mm in length. Inlet spout 34 and outlet spout 36 are affixed to the microfluidic device 30 at the respective ends of the cylindrical microchannel 32 to facilitate conveyance of fluid through cylindrical microchannel 32. Of course, in other embodiments, a plurality of cylindrical microchannels may be provided, and different materials may be used. Elastomers such as PDMS are particularly appealing because of their excellent biocompatibility, high patterning possibility, and excellent optical quality. These properties enable numerous applications that combine biological analysis, flow, and optical analysis including microfluidics and opto-fluidics applications.

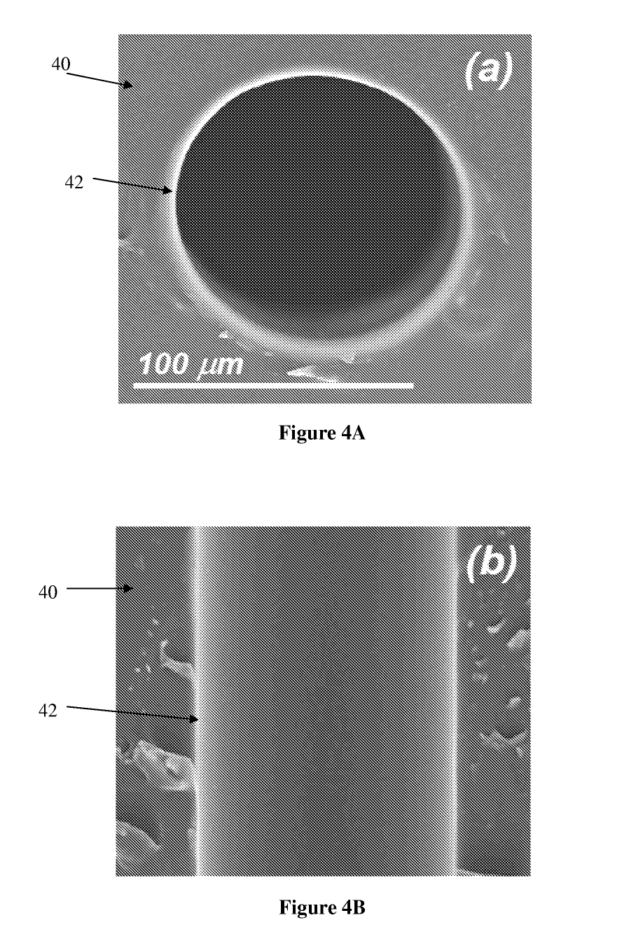

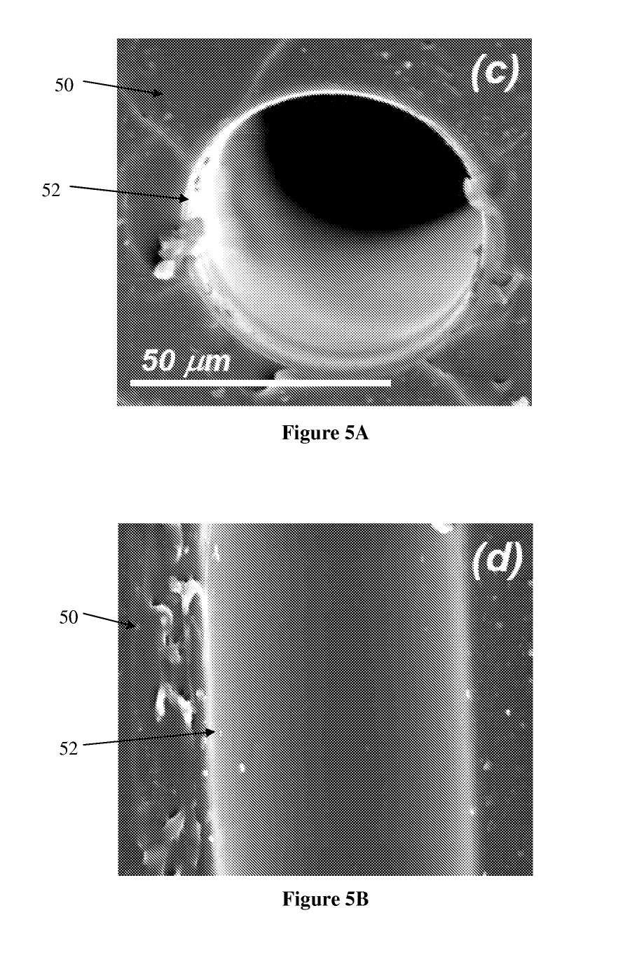

FIGS. 4A and 4B show scanning electron microscope (SEM) images of orthogonal and longitudinal sections, respectively, of a microfluidic device 40 with an embedded microchannel 42 having a diameter of approximately 125 μm. Similarly, FIGS. 5A and 5B show scanning electron microscope images of orthogonal and longitudinal sections of a microfluidic device 50 with an embedded microchannel 52 having a diameter of approximately 57 μm. These figures clearly show the regular, seamless, and smooth cylindrical channel that is manufactured within a microfluidic device fabricated in accordance with the method of the present invention. As explained, conventional methods of fabricating microfluidic devices that use soft-lithography techniques cannot produce the illustrated smooth structures of the microchannels or the circular cross-section.

Of course, the cylindrical microchannel may be implemented with different diameters by using different diameter elongated rods, such as the silica rods described above. Silicon fibers of different diameters may be used to realize these structures within the microfluidic devices. Moreover, a plurality of silica rods may be used to provide a plurality of cylindrical microchannels within the microfluidic devices. For example, the diameter of the cylindrical microchannel may be approximately 40 μm to 250 μm. Microchannels with diameters in this range are well-suited for manufacture using the method of the present invention as described above. Cylindrical microchannels having smaller diameters are formed in accordance with the present invention by custom drawing smaller diameter silica rods for use in the fabrication process. As outlined above, cylindrical microchannels with these smaller diameters are especially difficult to manufacture using conventional methods. Correspondingly, using the method of the present invention, microfluidic devices with cylindrical microchannels may be manufactured with a variety of lengths and widths using correspondingly-sized mounts upon which the microfluidic devices are formed.

A cylindrical microchannel with a diameter of approximately 40 μm to 250 μm is particularly effective for modeling vasculature, such as human capillaries, and for facilitating flow velocities of fluids between 3 and 5 mm/sec through microchannels that are commensurate with natural systems. In this regard, the functionality of the microfluidic device with a cylindrical microchannel formed in accordance with the present invention described above, and the suitability for applications in optically based flow cytometry, was examined by using the microfluidic device 30 of FIG. 3.

In particular, real-time video images of blood flowing through microchannel 32 were acquired by placing microfluidic device 30 of FIG. 3 on a modified microscope stage, and trans-illuminating the microfluidic device using 520 nm Philips Lumiled™ LEDs. Due to hemoglobin absorption, the green LED was found to provide a good contrast between the surrounding PDMS substrate and the microchannel channel 32 with the flowing red blood cells. Flow through the microfluidic device 30 was controlled by a mechanical syringe pump available from Harvard Apparatus. Images were captured with an Olympus 40×, 0.6 NA microscope objective and a monochrome CCD camera manufactured by Watec America Corporation, using a 30 mm tube lens. The images were recorded by a computer using a video capture card and NeoDVD software.

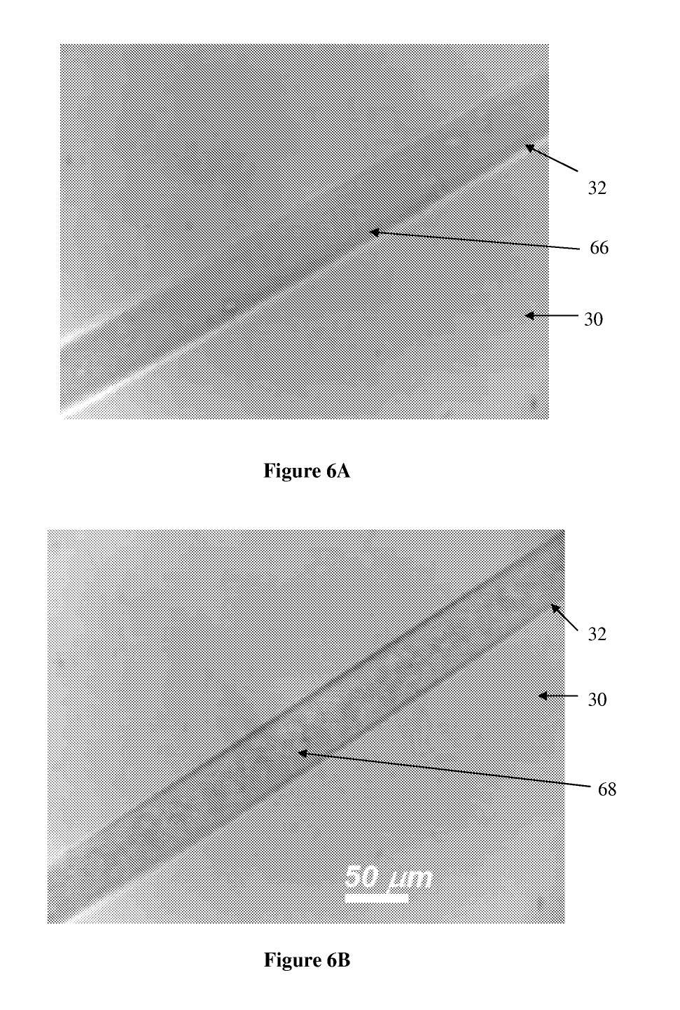

FIG. 6A shows an enlarged still frame from the video image of the microfluidic device 30 of FIG. 3 through which human blood was conveyed. As shown in FIG. 6A, the microfluidic device 30 includes a cylindrical microchannel 32 having a diameter of 57 μm and a length of approximately 300 mm. The 57 μm diameter is an appropriate size for modeling human vasculature. As can be seen in FIG. 6A, heparin 66 is visible in the blood flow through the cylindrical microchannel 32. Similarly, FIG. 6B shows erythrocytes 68 in the blood flowing through the cylindrical microchannel 32.

The flow of human red blood cells in the cylindrical microchannel was optically measured to assess the performance of microfluidic device 30. To demonstrate sustained flow through such a microchannel, red blood cells were labeled with Vybrant® DiD Molecular Probes™ from Invitrogen™, which is a lipophilic, fluorescent live cell stain that binds to cell membranes. Red blood cells (RBC) suspended in Dulbecco's Modified Eagle Medium (DMEM) from Hyclone company were incubated with a 55 μM Vybrant® DiD solution at 37° C. for 30 minutes and then washed 3 times to remove any excess dye. Then, 500 μl of the labeled red blood cells were added to a 20% hematocrit unstained red blood cell solution.

This blood suspension with DiD labeled cells was flowed through the microchannel 32 of the microfluidic device 30 using a mechanical syringe pump at a flow rate of 0.0009 cc/minute, resulting in a blood flow velocity of approximately 4.3 mm/second in the microchannel 32, thereby effectively modeling blood flow velocity found in vivo. The labeled cells were excited, and fluorescence was collected using a modified flow cytometry system based on confocal excitation and detection.

A HeNe laser was focused into a slit and imaged across the microfluidic channel using a microscope objective. As each DiD-labeled cell moved across the slit, it emitted a burst of fluorescence that was collected by the objective and imaged onto a mechanical slit in front of a photomultiplier tube (PMT). The fluorescence was sampled at a rate of 6.7 kHz using a data acquisition card and the resulting digitized signal was displayed in real-time and stored on the computer. Since the detection slit was confocal to the excitation slit, the PMT detected light predominantly from the focus of the objective. Moreover, a 670/40 nm bandpass emitter filter was placed in front of the detection slit to reduce the detection of backscattered excitation light, so that peaks in the digitized signal corresponded to fluorescence from DiD labeled cells excited by the HeNe slit.

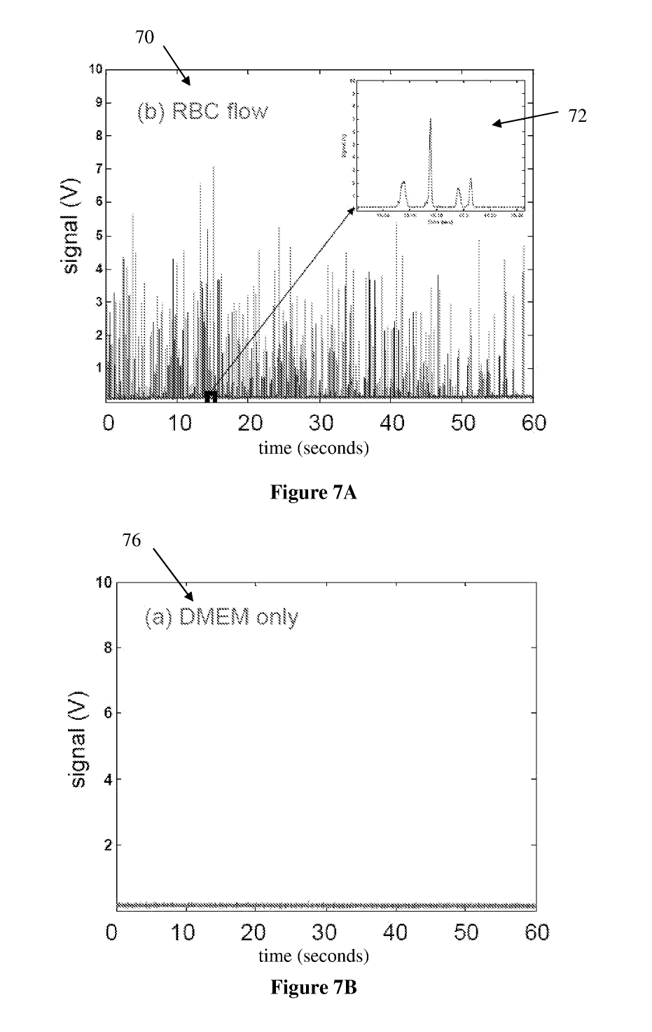

Fluorescence data was acquired for 60 seconds while flowing DMEM with the labeled red blood cell (RBC) solution. The results of the RBC flow during this time period are shown as the data in graph 70 of FIG. 7A. The Y-axis of graph 70 indicates the received fluorescence signal in volts, while the X-axis indicates time in seconds. Thus, the peaks shown in graph 70 of FIG. 7A represent detection of a labeled red blood cell moving across the detection slit where the signal is generated by cell fluorescence. A portion of the data that has been expanded in the time domain is shown in the inset 72 of FIG. 7A.

The actual flow of the red blood cells was also confirmed by flowing DMEM only into microchannel 32 of microfluidic device 30. Data acquired while flowing DMEM by itself, without added labeled red blood cells, is shown in graph 76 of FIG. 7B. As can be seen, the data of graph 76 shows no visible peaks in the digitized time traces. Thus, this data shown in graph 76 confirms the red blood cell flow within the microchannel 32 that was recorded in graph 70 of FIG. 7A. The successful performance of microfluidic device 30 in modeling vasculature, such as human capillaries, affirms its suitability for a number of applications such as cell sorting, specialized tubing for flow cytometry, and for biomedical applications in optical sensing.

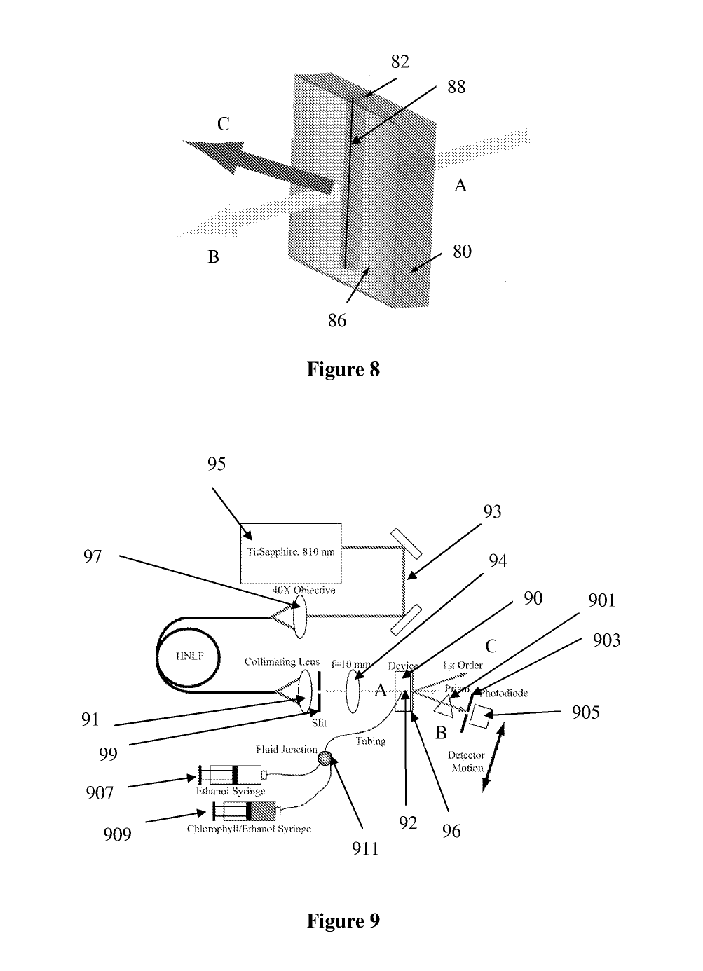

In another embodiment, a cylindrical microfluidic channel is incorporated in a planar, optofluidic integrated spectrometer. As shown in FIG. 8, the planar optofluidic integrated spectrometer device 80 includes microfluidic channel 82 suspended at a distance behind diffraction grating 86 all fabricated on a monolithic “chip” of siloxane polymer. Of course other polymers or biopolymers may be used depending upon the desired characteristics of the device. Light A is used to probe the absorption of a fluid 88 inside the microfluidic channel 82. Light A enters from the side of device 80, interacts with fluid 88 and propagates toward the transmission diffraction grating 86 where it is diffracted. Using a slit (not shown), the diffracted beam B, C is analyzed for transmitted power as a function of wavelength. In the example embodiment, supercontinuum light A was used to probe the device 80 and to perform spectroscopy of a chlorophyll solution, which displays its characteristic absorption spectrum. Essentially, device 80 can be thought of as a scanning grating spectrometer whose diffractive element 86 integrates microfluidic structures, such as microfluidic channel 82 and whose contents can, in turn, be spectrally analyzed by the diffractive structure element 86.

The device 80 may be fabricated using soft lithography in polydimethylsiloxane (PDMS) polymer. PDMS is chosen for its chemical stability, ease of handling, and high optical transparency, but other polymers or biopolymers may also be used, depending upon the desired characteristics of the device and the fluids that will be analyzed.

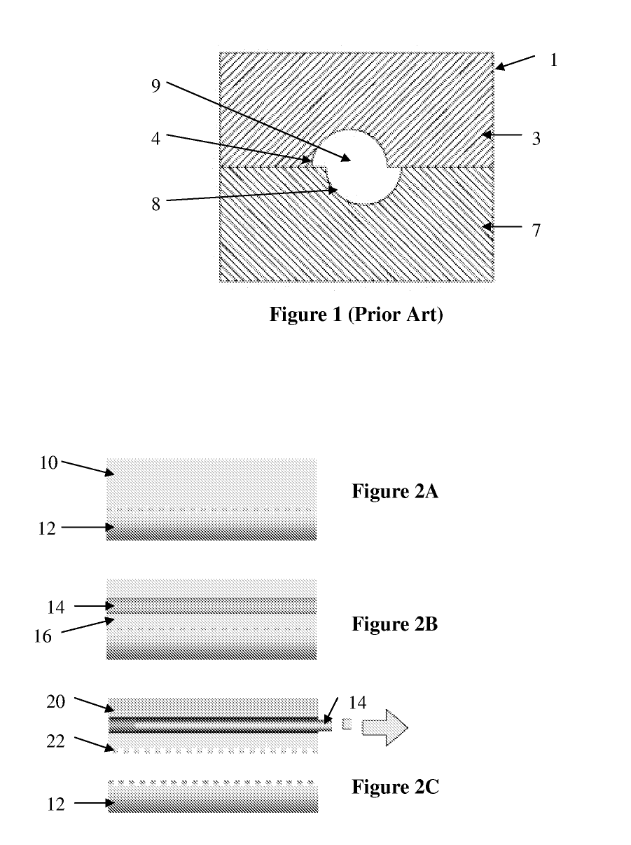

As described above with regard to FIGS. 1 and 2, the polymer is fabricated using a mold or substrate with which to impart patterns to the polymer as it dries. When the still-liquid polymer is poured upon the mold, it conformally fills it features and, once hardened, forms a replica of the mold surface. The mold used in this device is a ruled reflection grating from Thorlabs, Inc. with a groove density of 600 lines per mm. The grating is placed in an enclosure so that the ruled surface of the grating forms the bottom surface of the enclosure. A 250 μm diameter silica capillary is mounted 5 mm above the grating surface, running parallel to the lines of the grating. This capillary will, eventually, form the microfluidic channel of the device. The PDMS is mixed and degassed according to the manufacturer's instructions (Dow Corning 200 PDMS) and poured into the mould to a depth of 1 cm. The PDMS is cured, and the mold is removed leaving a 1 cm thick chip of PDMS with a microfluidic channel running through the middle and a diffraction grating on one side.

FIG. 9 illustrates a configuration in accordance with the present invention used to optically probe device 90. Supercontinuum light A is generated by a titanium sapphire laser 95 coupling 110 fs, 80 MHz pulses at a wavelength of 810 nm with average power of 1.8 W into 20 cm of silica high-Δ photonic crystal fiber (PCF) 93 with a coupling efficiency of ˜40% using a 25×, 0.5 NA microscope objective 97 on a 3-axis positioner (not shown). The supercontinuum light A generated spans the visible wavelength range and continues into the near infra-red.

The supercontinuum light A exits the PCF and is collimated using an aspheric collimating lens 91. While the lens 91 possesses chromatic aberration over the supercontinuum bandwidth, the comparatively narrow wavelength range utilized can be considered collimated. The beam of light A then passes through an imaging slit 99 with 1 mm width, which acts as a spatial filter, creating an image of the supercontinuum light that is rectangular and has the same directionality as the microfluidic channel 92. This image is focused onto the microfluidic channel 92 using a f=10 cm lens 94. This lens 94 and imaging slit width is chosen so that, at its focus, the probe light will be focused entirely within the microfluidic channel 92.

The device 90 itself is mounted on a quartz slide (not shown) and is oriented so that the microfluidic channel 92 and the transmission grating lines run vertically. The device 90 is aligned such that the incident beam of light A is normal to the surface of the quartz slide and device 90. Once the light A passes through the microfluidic channel 92 and is potentially absorbed, it is diffracted by the cast transmission grating 96. We examine the first diffraction order for spectroscopic variation.

A fluorite prism 901 is placed into the first order diffraction path to act as a selectivity filter between the diffracted orders. This allows light around the angle of the first diffraction order C to pass, but light of other orders (and angles) to be diffracted away from the first order C. It should be appreciated that the presence of the prism is not necessary if the pitch of the grating used is different as the higher lines/mm will disperse light more readily. The first diffraction order C is spectrally analyzed using a slit 903 with 0 5 mm width in front of a photodiode InGaAs detector 905, such as a Thorlabs DET410, for example. The output of the detector 905 is viewed on an oscilloscope (not shown). The entire slit/detector 903, 905 apparatus is traversed linearly on a micrometer driven translation stage perpendicular to optical beam direction at the center of the visible first order diffracted beam C. Readings from the detector 905 are read from the oscilloscope (not shown) as a function of position. The device 90 is calibrated by using a pair of 10 nm band pass filters with central wavelengths of 600 and 530 nm. With these filters inserted, a calibration function can be determined for the wavelength analyzed by the device 90 as a function of detector position. In this manner, a simple grating spectrometer is created that has an integrated, microfluidic sample chamber whose diffractive element was fabricated using soft lithography.

Additionally, microfluidic plumbing may be coupled to the device 90. Two syringes 907, 909 are coupled to the top of the device 90 using a stainless steel Y-junction 911 with 0.5 mm apertures attached using clear, silicone rubber tubing. One syringe 907 is filled with ethanol and the other syringe 909 is filled with a chlorophyll solution in ethanol. The output aperture of the microfluidic channel 92 has a stainless steel tube with 0.5 mm diameter, which is connected to a length of tube to transport away waste from the device 90. The diameter of the steel fittings used in comparison with the cast microfluidic channel 92 diameter ensures a water-tight fit for all practical fluid pressures. Fluids (not shown) are actuated through the device 90 by manually depressing the appropriate syringe 907, 909. Typically, two seconds of 0.25 mL/s fluid flow is used to ensure that the microfluidic channel 92 is cleaned of the previously occupying fluid.

Spectroscopy of the chlorophyll solution is performed using a background subtraction technique. First, a dark current of the device is taken without the supercontinuum source. Then, as a reference, the absorption spectrum of straight ethanol is taken using the supercontinuum source on. Finally, the chlorophyll solution is pumped into the device as described earlier, and the spectrum of the chlorophyll solution is taken with the previous reference subtracted numerically. The calibration procedure described above is performed periodically with pure ethanol in the device.

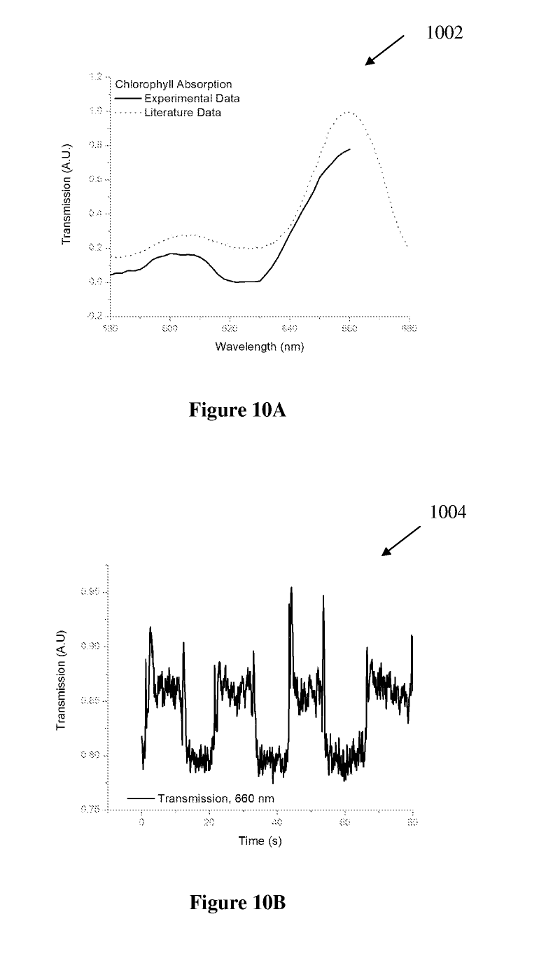

FIG. 10A shows a plot 1002 of the absorption spectrum of a chlorophyll solution in ethanol compared to values available from previous experiments. Over the bandwidth available to the device, the spectra appear to match quite closely. This facsimile of absorption spectra lends confidence to the design and operation of the device.

FIG. 10B also shows a plot 1004 of the temporal response of the device. Since the fluids there in can be actuated in temporal patterns, the temporal response of the device can also be measured. This is performed by tuning the wavelength of the spectrometer to 660 nm, the maximum absorption of chlorophyll in the red. Then, the detector signal is monitored temporally as the ethanol and the chlorophyll solution are alternately fed through the device. The pumping regime followed that described above, where one fluid was actuated for 2 sec at 0.25 mL/s then held steady for 8 seconds. After this time, the process is repeated for the next fluid. The modulation of the transmission at this wavelength is dependent upon the absorption of the species present. Also apparent is the 2 second transition region where the water and ethanol solution mix, creating a transient in the transmission.

The demonstrated embodiment realizes optofluidic tuning by combining microfluidic architecture with a diffractive optical element allowing spectral absorption in the channels to the analyzed. In this embodiment, an easily fabricated yet highly functional optofluidic device provides significant functionality in a compact package.

Furthermore, spectrally selective optical elements can be seamlessly incorporated into the fabrication method of the present invention so that additional compact and disposable opto-fluidic devices can be fabricated. In particular, optical functionality may be provided in the microfluidic device by casting the polymer on an appropriate optical mold such as other optical gratings. Similarly, the polymer or biopolymer may be cast onto other optical devices including a lens, a microlens array, a pattern generator, a beam reshaper, a mirror blank, or a glass slide. In such embodiments, when the optical mold is removed upon polymerization of the matrix solution, a multifunctional integrated device is provided that includes both an embedded cylindrical microchannel, and an optical element. Thus, the microfluidic device is ideally suited for various kinds of spectral flow studies. Additionally, a microfluidic device in accordance with the present invention can be further modified to incorporate doping agents within the uncured polymer matrix solution, thereby functionalizing the microfluidic devices to provide spectral detection capabilities. For example, the doping agents may include organic materials such as red blood cells, horseradish peroxidase, and phenolsulfonphthalein (phenol red), or a combination thereof. For instance, the microfluidic device doped with a doping agent such as phenol red causes color change when a specific fluid is conveyed through the cylindrical microchannel formed in the microfluidic device.

The organic material can also be a nucleic acid, a dye, a cell, an antibody, as described further in Appendix I, enzymes, for example, peroxidase, lipase, amylose, organophosphate dehydrogenase, ligases, restriction endonucleases, ribonucleases, DNA polymerases, glucose oxidase, laccase, cells, viruses, bacterias, proteins, peptides for molecular recognition, small molecules, drugs, dyes, amino acids, vitamins, antixoxidants, plant cells, mammalian cells, and the like, DNA, RNA, RNAi, lipids, nucleotides, aptamers, carbohydrates, optically-active chromophores ncluding beta carotene or porphyrins, light emitting organic compounds such as luciferin, carotenes and light emitting inorganic compounds, chemical dyes, antibiotics, yeast, antifungals, antivirals, and complexes such as hemoglobin, electron transport chain coenzymes and redox components,light harvesting compounds such as chlorophyll, phycobiliproteins, bacteriorhodopsin, protorhodopsin, and porphyrins and related electronically active compounds, or a combination thereof.

By providing a method for reliably and cost-effectively manufacturing microfluidic devices with cylindrical microchannels, diagnostic and medical applications are enabled. In particular, such microfluidic devices are biomedically significant in enabling “lab-on-chip” tools and diagnostic devices that provide convenience and functionality in a small device.

The present invention provides a microfluidic device having one or more microchannels. The present invention provides a method for forming one or more cylindrical microchannels. It should also be evident that the present invention provides a method for fabricating a microfluidic device with one or more cylindrical microchannels that can be used to model microvasculature of animals and humans.

The foregoing description of the aspects and embodiments of the present invention provides illustration and description, but is not intended to be exhaustive or to limit the invention to the precise form disclosed. Those of skill in the art will recognize certain modifications, permutations, additions, and combinations of those embodiments and features are possible in light of the above teachings or may be acquired from practice of the invention. Therefore, the present invention also covers various modifications and equivalent arrangements and methods that fall within the purview of the appended claims.

What is claimed is:

1. A method of manufacturing a microfluidic device having at least one cylindrical microchannel comprising:
providing a substrate;
casting an uncured polymer matrix solution onto said substrate;
embedding an elongated rod in said uncured polymer matrix solution;
curing said polymer matrix solution to form a solidified body of said microfluidic device; and
extracting said elongated rod to form said cylindrical microchannel in said solidified body.

2. The method of claim 1, wherein said elongated rod is a silica rod.

3. The method of claim 2, wherein said silica rod has a diameter between approximately 40 μm and 250 μm, inclusive.

4. The method of claim 3, wherein said silica rod has a diameter between approximately 57 μm and 125 μm, inclusive.

5. The method of claim 1, wherein said polymer matrix solution includes polydimethylsiloxane (PDMS).

6. The method of claim 1, wherein said polymer matrix solution includes a biopolymer.

7. The method of claim 6, wherein said biopolymer is selected from a group consisting of chitosan, collagen, gelatin, agarose, chitin, polyhydroxyalkanoates, pullan, starch (amylose amylopectin), cellulose, hyaluronic acid, and related biopolymers, or a combination thereof.

8. The method of claim 6, wherein said biopolymer is silk.

9. The method of claim 1, wherein said polymer matrix solution is an aqueous silk fibroin solution having approximately 1.0 wt % to 30 wt % silk, inclusive.

10. The method of claim 9, wherein said aqueous silk fibroin solution has approximately 8.0 wt %.

11. The method of claim 1, wherein said curing said polymer matrix solution includes applying heat to said uncured polymer matrix solution.

12. The method of claim 1, further comprising:
coating said silica rod with a surfactant solution.

13. The method of claim 1, further comprising
forming an optical element on a surface of said microfluidic device.

14. The method of claim 13, wherein said substrate is a template for said optical element.

15. The method of claim 14, wherein said optical element is at least one of a lens, a microlens array, an optical grating, a pattern generator, a beam reshaper, a mirror blank, and a glass slide.

16. The method of claim 1, further comprising:
adding a doping agent to said uncured polymer matrix solution.

17. The method of claim 16, wherein said doping agent is selected from a group consisting of red blood cells, horseradish peroxidase, and phenolsulfonphthalein, or a combination thereof.

18. The method of claim 16, wherein said doping agent is selected from a group consisting of a nucleic acid, a dye, a cell, an antibody, enzymes, for example, peroxidase, lipase, amylose, organophosphate dehydrogenase, ligases, restriction endonucleases, ribonucleases, DNA polymerases, glucose oxidase, laccase, cells, viruses, proteins, peptides, small molecules, drugs, dyes, amino acids, vitamins, antixoxidants, DNA, RNA, RNAi, lipids, nucleotides, aptamers, carbohydrates, chromophores, light emitting organic compounds such as luciferin, carotenes and light emitting inorganic compounds, chemical dyes, antibiotics, antifungals, antivirals, light harvesting compounds such as chlorophyll, bacteriorhodopsin, protorhodopsin, and porphyrins and related electronically active compounds, or a combination thereof.

19. The method of claim 1, further comprising:
suspending said elongated rod over said substrate.

20. A microfluidic device comprising:
a polymer body; and
at least one cylindrical microchannel in said polymer body, said cylindrical microchannel having a diameter between approximately 40 μm and 250 μm, inclusive.

21. The microfluidic device of claim 20, wherein said cylindrical microchannel has a diameter between approximately 57 μm and 125 μm, inclusive.

22. The microfluidic device of claim 20, wherein said polymer body includes polydimethylsiloxane (PDMS).

23. The microfluidic device of claim 20, wherein said polymer body includes a biopolymer selected from a group consisting of chitosan, collagen, gelatin, agarose, chitin, polyhydroxyalkanoates, pullan, starch (amylose amylopectin), cellulose, hyaluronic acid, and related biopolymers, or a combination thereof.

24. The microfluidic device of claim 20, wherein said polymer body includes a silk biopolymer.

25. The microfluidic device of claim 20, wherein said polymer body includes an optical element on a surface thereof.

26. The microfluidic device of claim 20, wherein said optical element is at least one of a lens, a microlens array, an optical grating, a pattern generator, a beam reshaper, a mirror blank, and a glass slide.

27. The microfluidic device of claim 20, wherein said polymer body includes a doping agent.

28. The microfluidic device of claim 27, wherein said doping agent is selected from a group consisting of red blood cells, horseradish peroxidase, and phenolsulfonphthalein, or a combination thereof.

29. The microfluidic device of claim 27, wherein said doping agent is selected from a group consisting of a nucleic acid, a dye, a cell, an antibody, enzymes, for example, peroxidase, lipase, amylose, organophosphate dehydrogenase, ligases, restriction endonucleases, ribonucleases, DNA polymerases, glucose oxidase, laccase, cells, viruses, proteins, peptides, small molecules, drugs, dyes, amino acids, vitamins, antixoxidants, DNA, RNA, RNAi, lipids, nucleotides, aptamers, carbohydrates, chromophores, light emitting organic compounds such as luciferin, carotenes and light emitting inorganic compounds, chemical dyes, antibiotics, antifungals, antivirals, light harvesting compounds such as chlorophyll, bacteriorhodopsin, protorhodopsin, and porphyrins and related electronically active compounds, or a combination thereof.

30. An optofluidic spectrometer comprising:
a polymer body;
a diffraction grating integrated with said polymer body; and
at least one cylindrical microchannel in said polymer body, said cylindrical microchannel having a diameter between approximately 40 μm and 250 μm, inclusive, and behind said diffraction grating on said polymer body.

31. The optofluidic spectrometer of claim 30, wherein said polymer body is a siloxane polymer chip.

32. The optofluidic spectrometer of claim 31, wherein said siloxane polymer chip includes polydimethylsiloxane (PDMS).

33. The optofluidic spectrometer of claim 30, wherein said polymer body is a biopolymer.

34. The optofluidic spectrometer of claim 33, wherein said biopolymer is silk.

35. A method of probing absorption of a fluid in a microfluidic channel comprising:
transmitting light through a polymer body, wherein said polymer body includes said microfluidic channel containing said fluid and a diffraction grating;
absorbing at least one wavelength of said light in said fluid;
diffracting said light with said diffraction grating;
analyzing said diffracted light for transmitted power as a function of wavelength with a slit; and
characterizing said fluid based upon said analysis of said diffracted light.
